(19) United States
(12) Patent Application Publication
(10) Pub. No.: US 2010/0105637 A1
(43) Pub. Date: Apr. 29, 2010

(54) Drug-surfactant complexes for sustained release

(76) Inventor: Cherng-ju Kim, Little Rock, AR (US)

Correspondence Address: HUGH MCTAVISH;MCTAVISH PATENT FIRM, 429 BIRCHWOOD COURTS, BIRCHWOOD, MN, 55110, US

(21) Appl. No.: 12/589,327

(22) Filed: Oct. 22, 2009

Related U.S. Application Data
(62) Division of application No. 11/207,126, filed on Aug. 17, 2005.

(51) Int. Cl. A61K 31/375; A61K 31/554; A61K 31/138; A61K 31/137; A61K 31/135; A61K 31/4365; A61K 31/4422; A61K 31/445; A61K 31/40; A61K 31/435; A61K 31/47; A61K 31/22; A61K 31/66; A61K 31/197; A61P 9/10

(52) U.S. Cl. 514/89; 514/211.07; 514/651; 514/654; 514/647; 514/649; 514/301; 514/356; 514/317; 514/423; 514/338; 514/311; 514/510; 514/103; 514/567

(57) ABSTRACT

The invention involves sustained-release pharmaceutical compositions containing a water-soluble ionic small molecule pharmaceutical agent complexed with an oppositely charged surfactant, particularly a natural bile surfactant. The complexes are sustained-release ionic complexes. The complexes release the ionic pharmaceutical agents into aqueous solution slowly and with zero-order kinetics. Thus, they can be formulated into sustained-release pharmaceutical compositions.

### BACKGROUND

Most drugs are administered in formulations that dissolve quickly in the gastrointestinal tract and are absorbed quickly into the blood stream. Thus, the administered dose is quickly dissipated and the concentration of the drug in the blood stream rises rapidly and then falls rapidly. This can necessitate frequent dosing. This raises the risk that patients will not comply with the dosing instructions, forgetting to take doses at the appropriate times or refusing to take all of their prescribed doses because of inconvenience. In addition, even when patients comply with the dosing instructions, the concentration of the drug in the blood stream will rise and fall throughout the day.

Sustained-release formulations avoid or lessen these problems. They decrease the number and frequency of doses and result in steadier concentrations of the drug in the blood stream.

New sustained-release formulations of pharmaceutical agents are needed. New methods of preparing sustained-release pharmaceutical agents and prolonging the release of pharmaceutical agents in the body are needed. Preferably these methods would result in formulations that release a constant amount of agent per unit time until all agent is released, i.e., have zero-order kinetics.

### SUMMARY

The inventors have discovered that water-soluble ionic pharmaceutical agents form complexes with oppositely charged ionic surfactants, such as anionic bile surfactants. The complexes dissociate slowly to release the pharmaceutical agents in aqueous solutions containing salts. The release kinetics are close to zero order. The release kinetics can be made slower and even closer to pure zero order by formulating the complexes with sustained-release polymers or fillers, such as hyroxypropylmethylcellulose. Thus, complexes between ionic pharmaceutical agents and oppositely charged ionic surfactants are effective sustained-release formulations of the pharmaceutical agents.

Bile salt anions are particularly favored surfactants for use in the invention because they are native to the body and thus are unlikely to induce any adverse reaction.

Accordingly, the invention provides a pharmaceutical composition that includes: (a) a sustained-release ionic complex containing (i) a cationic non-peptidyl small molecule pharmaceutically active agent having a molecular weight of less than 2,000 and a solubility in water of at least 2 mg/ml, complexed with (ii) a bile anionic surfactant; in combination with (b) a pharmaceutically acceptable diluent or carrier.

Another embodiment of the invention provides a pharmaceutical composition that includes: (a) a sustained-release ionic complex containing (i) an ionic small molecule pharmaceutically active agent having a molecular weight of less than 2,000 and a solubility in water of at least 2 mg/ml, complexed with (ii) an oppositely charged ionic surfactant; (b) in combination with a pharmaceutically acceptable diluent or carrier. In this embodiment the pharmaceutical composition releases the ionic pharmaceutically active agent into an aqueous solution containing salts with zero-order kinetics; and the sustained-release ionic complex is formed by a process comprising contacting the ionic small molecule pharmaceutically active agent with the oppositely charged ionic surfactant in aqueous solution to form the sustained-release ionic complex as a solid precipitate.

Another embodiment of the invention provides a method of preparing a sustained-release medicament involving: (a) contacting an ionic small molecule pharmaceutically active agent, having a molecular weight of less than 2,000 and a solubility in water of at least 2 mg/ml, with an oppositely charged ionic surfactant in aqueous solution to form a sustained-release ionic complex between the active agent and the surfactant; and (b) formulating the sustained-release ionic complex into a sustained-release medicament.

Another embodiment of the invention provides a method of preparing a sustained-release medicament involving: contacting a cationic small molecule pharmaceutically active agent, having a molecular weight of less than 2,000 and a solubility in water of at least 2 mg/ml, with an anionic bile surfactant to form a sustained-release ionic complex between the active agent and the surfactant; and formulating the sustained-release ionic complex into a sustained-release medicament.

Another embodiment of the invention provides a method of sustaining release of a pharmaceutical agent comprising: obtaining a pharmaceutical composition of the invention; and administering the pharmaceutical composition to a subject afflicted with a condition susceptible to treatment with the pharmaceutically active agent of the pharmaceutical composition.

### BRIEF DESCRIPTION OF THE DRAWINGS

FIG. 1 shows a plot of fractional release of diltiazem from diltiazem-HCl and diltiazem-deoxycholate against time.

FIG. 2 shows a plot of fractional release of diltiazem from diltiazem-HCl and diltiazem-deoxycholate each in a polymeric carrier against time.

FIG. 3 shows a plot against time of fractional release of diltiazem from diltiazem-deoxycholate in various polymeric carriers.

FIG. 4 shows a plot against time of fractional release of four drugs complexed with deoxycholate in an HPMC carrier.

FIG. 5 shows a plot of diltiazem release from tablets of diltiazem-deoxycholate complex with HPMC in various ratios of HPMC to drug complex.

FIG. 6 shows a plot of diltiazem release from 1, 2, or 3 tablets of diltiazem-deoxycholate with 50% HPMC.

FIG. 7 shows a plot of fractional release of diltiazem release from a tablet of diltiazem-taurodeoxycholate against time.

### DETAILED DESCRIPTION

### Definitions:

The term “surfactant” as used herein refers to an amphipathic substance containing a polar head group and non-polar tail. Surfactants are soluble in water and form organized spherical structures called micelles containing several molecules of the surfactant in aqueous solutions under at least some conditions. They can solubilize at least some hydrophobic substances under some conditions in aqueous solutions.

The term “bile surfactant” refers to a surfactant having a steroidal hydrophobic group.

The term “sustained-release ionic complex” refers to an ionic complex between a small molecule pharmaceutically active agent and an oppositely charged ionic surfactant that in aqueous solution releases the active agent into solution more slowly than it is released from the corresponding conventional salt of the active agent with a small oppositely charged ion such as chloride or sodium.

### Description:

The invention involves sustained-release medicaments containing an ionic pharmaceutically active agent complexed with an oppositely charged ionic surfactant.

The complexes can be formed by preparing a solution of a salt of the ionic active agent, e.g., a chloride salt of a cationic active agent or a sodium or potassium salt of an anionic active agent, in water, and preparing a solution of a simple salt of the ionic surfactant, e.g., the sodium salt of an anionic surfactant such as cholate, in water. Then the two aqueous solutions are mixed, and a complex of the active agent with the surfactant forms and precipitates.

The active agent is preferably a small molecule active agent with a molecular weight of less than 2,000. In another embodiment, the ionic active agent's molecular weight is less than 1,000. These molecular weights refer to the molecular weight of the ionic species of the active agent without a counter-ion.

In particular embodiments, the active agent is non-peptidyl. By “non-peptidyl” it is meant that less than 50% of the weight of the agent is units of the 20 naturally occurring amino acids in either the D or L stereochemistry. In specific embodiments, less than 10% of the weight of the agent is units of the 20 naturally occurring amino acids in either the D or L stereochemistry.

One important aspect of the invention is that the complexes tend to release the active agent with close to zero-order kinetics. This results in release of a relatively constant amount of drug per unit of time. (In pure zero-order kinetics, a constant amount of the active agent is released per unit of time until all of the active agent is released.) Thus, in a particular embodiment of the invention, the sustained-release medicament releases the ionic active agent into solution with zero-order kinetics in an aqueous solution containing salts. The solution may be at an enteric pH, e.g., approximately 1.5, as in the stomach, or approximately 7-8, as in the small intestine. Preferably, the sustained-release medicament releases the ionic active agent into solution with zero-order kinetics in aqueous solution containing salts at both approximately pH 1.5 and approximately pH 7-8. In another embodiment, the complex (without a sustained-release polymer or other components that might be present in some of the formulations of medicaments of the invention) releases the ionic active agent into solution with zero-order kinetics in an aqueous solution containing salts. By “zero-order kinetics” it is meant that the kinetics of release of the ionic active agent fit more closely to zero-order kinetics than to first order kinetics over the time course of release covering release of at to least 50% of the active agent. The sustained-release complexes of the invention typically release the ionic active agent into an aqueous solution containing salts with kinetics much closer to zero order than to first order. The deviations from pure zero-order kinetics are thought to be primarily because of tablet geometry. Formulated in a slab geometry, it is believed the sustained-release complexes would release active agent with almost pure zero-order kinetics.

The aqueous solution into which the sustained-release complexes release the active agent must contain some salt in order to provide a counterion to replace the surfactant and solubilize the ionic active agent.

The ionic pharmaceutically active agents in the complexes of the invention preferably have a solubility in water of at least 2 mg/ml, in some embodiments at least 40 mg/ml, and in some embodiments at least 100 mg/ml. These solubility levels refer to the solubility of a simple salt of the ionic active agent with a small counterion, such as chloride, sodium, sulfate, or calcium.

In particular embodiments, the surfactant is a naturally occurring surfactant in mammals (e.g., humans). Naturally occurring surfactants have the advantage of being unlikely to produce adverse reactions. The path to regulatory approval of drug preparations containing natural surfactants is also likely to be simpler than preparations containing artificial surfactants. Examples of naturally occurring surfactants include naturally occurring bile surfactants that are secreted by the gall bladder into the digestive system. Some examples include deoxycholate, cholate, chenodeoxycholate, ursodeoxycholate, and lithocholate; and their taurine and glycine conjugates taurocholate, glycholate, taurodeoxycholate, glycodeoxycholate, taurochenodeoxycholate, glycochenodeoxycholate, tauroursodeoxycholate, glycoursodeoxycholate, taurolithocholate, and glycolithocholate.

Another class of naturally occurring surfactants suitable for use in the invention is carboxylate anions of the naturally occurring fatty acids. These include oleate, palmitate, and stearate. Other examples include the carboxylate anions of myristic acid, arachidic acid, palmitoleic acid, linoleic acid, alpha-linolenic acid, and arachidonic acid. The fatty acids are generally of the formula (C₅-C₂₅)alkyl-COOH, wherein alkyl may include 0-3 unsaturated carbon-carbon bonds.

In a particular embodiment, the pharmaceutically active agent is cationic and the surfactant is anionic. In another embodiment, the pharmaceutically active agent is anionic and the surfactant is cationic.

In a particular embodiment where the surfactant is anionic, the surfactant is a bile anionic surfactant. It may be a natural bile surfactant of mammals, (e.g., of humans). Or it may be a synthetic bile surfactant (synthesized completely synthetically or semi-synthetically using natural bile or steroidal starting materials).

In particular embodiments, the bile surfactant is a compound of formula I:

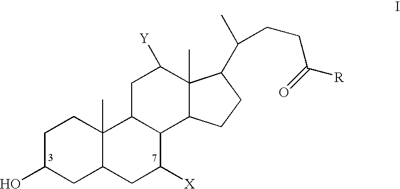

wherein Y is OH or H, X is OH or H, and R is any suitable anionic group of from 1 to 200 atoms.

The preferred stereochemistry of a compound of formula I is as shown below:

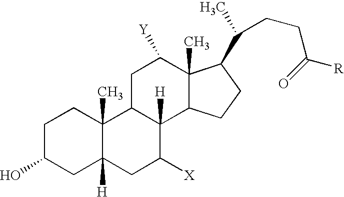

In preferred embodiments of a compound of formula I, R is —O⁻, —NHCH₂CO₂⁻, or —NHCH₂CH₂SO₃⁻.

Particular anionic bile surfactants natural in humans and suitable for use in the invention include deoxycholate, cholate, chenodeoxycholate, ursodeoxycholate, lithocholate, taurocholate, glycholate, taurodeoxycholate, glycodeoxycholate, taurochenodeoxycholate, glycochenodeoxycholate, tauroursodeoxycholate, glycoursodeoxycholate, taurolithocholate, and glycolithocholate. A synthetic bile surfactant suitable for use in the invention is 4′-amino-7-benzamide-taurocholate (BATC).

Other particular anionic bile surfactants suitable for use in the invention include those bile surfactants of formula I sulfated at the 3-hydroxyl, e.g., sulfolithocholate.

Some examples of cationic surfactants suitable for use with anionic pharmaceutical agents in the invention include hexadecylpyridinium and hexadecyltrimethylammonium, and benzalkonium. Benzalkonium is (C₁₂-C₁₆)alkylbenzyldimethylammonium

In specific embodiments, the cationic surfactant is of the formula NR₃⁺—(C₆-C₂₄)alkyl, wherein alkyl may include 0-3 unsaturated carbon-carbon bonds, and each R is independently H or CH₃.

One embodiment of the invention involves a method of preparing a sustained-release medicament involving contacting the ionic small molecule pharmaceutically active agent with the oppositely charged surfactant to form a sustained-release ionic complex between the active agent and the surfactant.

In particular embodiments, the contacting is in aqueous solution and the ionic sustained-release complex forms as a precipitate. The precipitate can form immediately or can form upon evaporating part or all of the solvent.

The precipitate of the sustained-release ionic complex can be redissolved in a solvent, for instance an organic solvent, along with a polymer matrix, such as hydroxypropylmethylcellulose or polylactic acid-polyglycolic acid copolymer. The solvent can then be removed from the mixture of ionic complex and polymer to entrap the sustained-release ionic complex uniformly distributed in a polymer matrix. The solvent can be evaporated in a mold to form an implant or a tablet, or can be evaporated by spray drying to form uniform particles of polymer matrix with entrapped active agent-surfactant ionic complex.

In another embodiment, the precipitated sustained-release ionic complex is formulated into a sustained-release medicament without redissolution in a solvent with a polymer matrix and precipitation in the polymer matrix. The precipitated sustained-release ionic complex can be mixed as a solid with excipients, including, e.g., a sustained-release polymer, and pressed into tablets.

As the above indicates, the pharmaceutical compositions of the invention may include in addition to the complex between the ionic pharmaceutically active agent and an oppositely charged surfactant a pharmaceutically acceptable diluent or carrier. The diluent or carrier can include a sustained-release agent—that is, an agent that helps to sustain release of pharmaceutically active agents, such as a sustained-release polymer, e.g., hydroxypropylmethylcellulose (HPMC).

Thus, in one embodiment of the method of preparing a sustained-release medicament, after forming the sustained-release complex between the ionic active agent and the ionic surfactant, the step of formulating the sustained-release complex into the sustained-release medicament involves mixing or coating the sustained-release complex with a sustained-release agent, such as a sustained-release polymer filler or coating to form a sustained-release medicament.

Particular sustained-release polymers for use in formulating the medicaments include HPMC, polethylene oxide, hyroxypropylcellulose, hydroxyethylcellulose, methylcellulose, a polysaccharide (e.g., cellulose or starch), and poly(acrylic acid) (CARBOMER™).

In particular embodiments, the pharmaceutical compositions do not include a polymer matrix that slows release of the pharmaceutically active agent from the sustained-release complex.

In particular embodiments where the ionic pharmaceutically active agent is cationic, the pharmaceutically active agent is diltiazem, propranolol, verapamil, lebatalol, setraline, venlafaxine, clopidogrel, amlodipine, fexofenadine, or bupropion. The sustained-release complexes are typically formed using the conventional salts of these agents, namely diltiazem HCl, propranolol HCl, verapamil HCl, lebatalol HCl, setraline HCl, venlafaxine HCl, clopidogrel bisulfate, amlodipine besylate, fexofenadine HCl, and bupropion HCl.

In particular embodiments where the ionic pharmaceutically active agent is anionic, the pharmaceutically active agent is atorvastatin, esomerprazole, montelukast, pravastatin, alendronate, levothyroxine, or risedronate. The sustained-release complexes are typically formed using the conventional salts of theses agents, namely atorvastatin calcium, esomerprazole magnesium, montelukast sodium, pravastatin sodium, alendronate sodium, levothyroxine sodium, and risedronate sodium.

Typically, the pharmaceutical compositions are formulated for oral administration.

The pharmaceutical compositions containing the complexes of an ionic pharmaceutically active agent with an oppositely charged ionic surfactant can be formulated with other agents that are conventionally used for sustaining release. Many of these are reviewed for instance, in De Haan, P. et al., 1984, Pharmaceutisch Weekblad Scientific Edition 6:57-67. These include fatty alcohols and fatty acid esters, including glyceryl monostearate and beeswax as coating materials in tablets and pellets of capsules (Blythe, U.S. Pat. Nos. 3,344,029 and 2,738,303). Another approach uses a coating membrane that impedes diffusion. This may be composed of ethylcellulose, other cellulose derivatives, or polymers of the polymethacrylate type. (Dreher, 1975, Pharmacy International 1(2):3. Lippold, B. C. et al., 1982, Pharm. Ind. 44:735. Reese, U.S. Pat. No. 3,437728.)

Slow release coated particles can be compressed into tablets (Juslin, M. et al. 1980, Pharm. Ind. 42:829).

The sustained-release complexes of the invention may be suspended in a fat or wax or a fat-wax mixture, by e.g., aqueous dispersion, spray congealing, or conventional granulating methods. (Kawashima, Y. et al., 1981, J. Pharm. Sci. 70:913. Robinson U. S. Pat. No. 3577514. John, P. M. et al., 1968, J. Pharm. Sci. 57:584. Wiseman, E. H. et al., 1968, J. Pharm. Sci. 57:1535.).

A particularly preferred technology for use with the present sustained-release complexes involves mixing with a polymer matrix. The release in this case is based on leaching through the pores of the matrix. The polymer matrix is typically an insoluble inert plastic (e.g., polyvinyl acetate, polyvinyl chloride, ethylcellulose, paraffin, or hydroxypropyl cellulose). (Georgakopoulos, P. P. et al., 1981 Acta Pharm. Techn. 27(4):231. Kala, H. et al., 1980. Pharmazie 35:418. Fryklof, L. E., 1960, Brit. Patent 808014. Sannerstedt, R., 1960, Acta Med Scand. 167:245.) The polymer matrix may be slowly eroding to expose the sustained-release complexes of the invention to the aqueous environment in vivo.

Other preferred polymer matrixes for use in the invention include hydrophilic polymers such as HPMC, carboxyvinyl polymers, acrylic acid copolymers, poly(lactic acid) and copolymers of lactic acid and glycolic acid. (Christenson, G. L. et al., 1962, U.S. Pat. No. 3,065,143. Huber, H. E. et al., 1966, J. Pharm. Sci. 55:974.) Polymers of lactic acid and glycolic acid are biodegradable and degrade to innocuous natural products.

Generally the sustained-release complexes provide adequate control over the release rate on their own, so that other mechanisms of controlling or slowing the release need not be incorporated into the pharmaceutical compositions and the pharmaceutical compositions need not be encased in devices or barriers that slow or control release. Thus, in particular embodiments, the pharmaceutical composition does not comprise a water-insoluble wall encasing or partially encasing the sustained-release complex.

The sustained-release complexes of the invention can be formulated as pharmaceutical compositions and administered to a mammalian host, such as a human patient in a variety of forms adapted to the chosen route of administration. Typically, the sustained-release complexes of the invention are formulated for oral administration. But the complexes can also be given by intramuscular injection. They can also be used in implanted sustained-release formulations or devices.

Thus, the complexes may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the complexes containing the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 100% or about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the complexes of the pharmaceutically active agent with an ionic surfactant, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed.

Generally, the concentration of the complexes in a pharmaceutical composition will be from about 0.1 to 100 wt-%, in some embodiments 0.1-40 wt-% or about 0.5-25 wt-%.

The invention will now be illustrated by the following non-limiting examples. They are intended to illustrate the invention but not limit the its scope.

### Examples

### Example 1

Diltiazem-HCl was dissolved in water at 5% w/v. Sodium deoxycholate was separately dissolved in water (5% w/v). The diltiazem and deoxycholate solutions were mixed. A precipitate of diltiazem-deoxycholate complex formed. The precipitate was removed and formulated into tablets (150 mg tablet, 0.3125 inches diameter) by a punch/die and a Carver press under 3000 pounds. No binders or excipients were added.

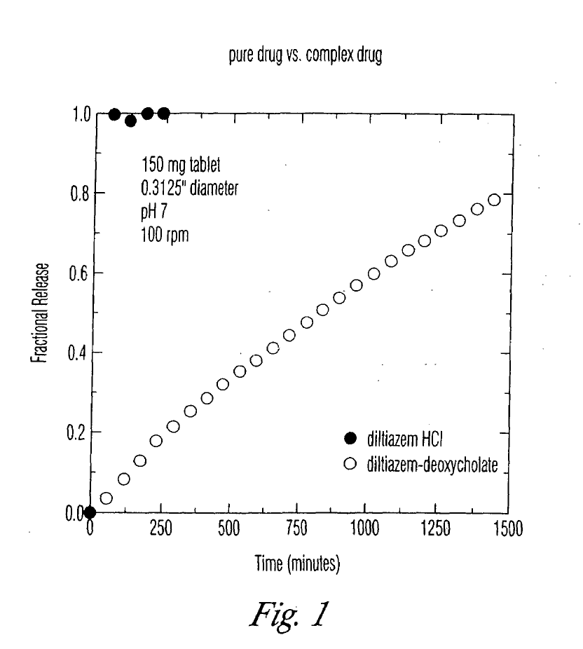

The tablets were placed into pH 7.0 water and stirred with a stirrer at 100 rpm. Fractional release was determined by uv/vis spectroscopy of the aqueous solution at 278 nm to measure the concentration of diltiazem in solution. The results are shown in FIG. 1. At the first time point, all diltiazem from the diltiazem-HCl was released. Diltiazem from diltiazem-deoxycholate released slowly over a time period of over 1500 minutes with kinetics close to zero-order. It is believed that the deviations from pure zero-order kinetics are due to tablet geometry. Formulated in a slab geometry, it is believed the complex will release active agent with almost pure zero-order kinetics.

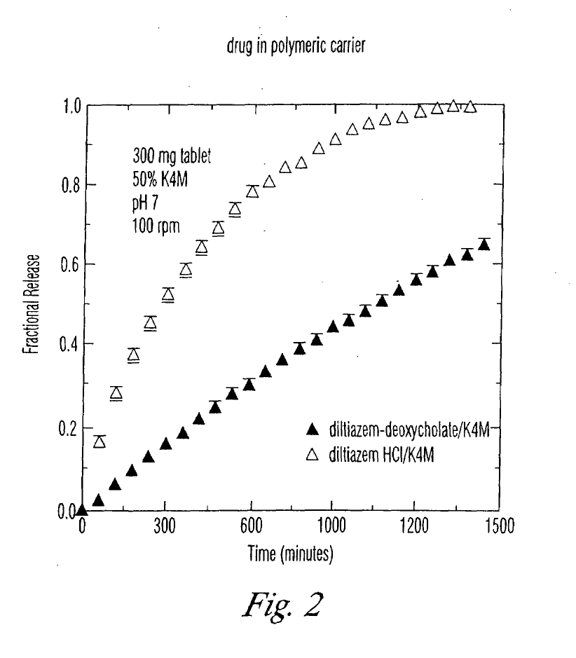

Next, diltiazem-HCl and diltiazem-deoxycholate were each formulated into 300 mg tablets containing 50% hydroxypropylmethylcellulose (HPMC) K4M. The tablets were placed in pH 7.0 water and stirred at 100 rpm, and fractional release was measured as above. The results are shown in FIG. 2. The active agent was released from diltiazem-HCl/K4M with apparent first order kinetics. Approximately half the agent was released in 400 minutes. In contrast, diltiazem was released from the diltiazem-deoxycholate//K4M tablets more slowly and with zero-order kinetics.

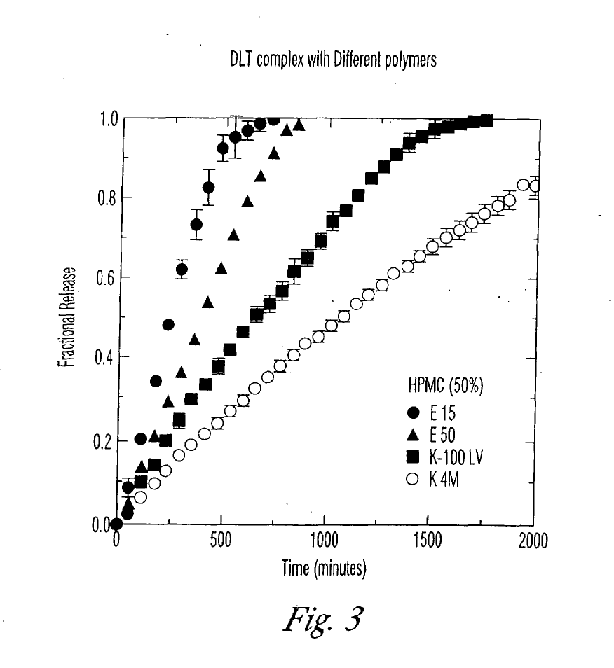

Diltiazem-deoxycholate was formulated into 300 mg tablets with 50% HPMC using different types of HPMC and fractional release was measured. The results are shown in FIG. 3. The release with all the HPMCs showed zero-order kinetics. The order of rate of release was E15>E50>K-100LV>K4M.

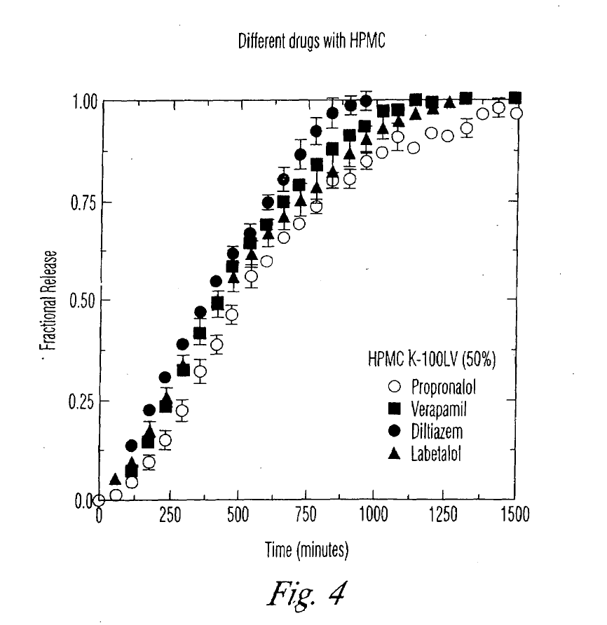

Next, four cationic drugs—propronalol, verapamil, diltiazem, and labetalol—were complexed with deoxycholate. The complexes were formulated into tablets with 50% HPMC K-100LV. Fractional release was measured by uv/vis spectroscopy and the results are shown in FIG. 4. All of the drugs released with substantially zero-order kinetics. Complete release of each of the drugs took approximately 1000 minutes. The graph of fractional release versus time showed slightly sigmoidal behavior. The initial small time lag for release is thought to be due to time for water absorption.

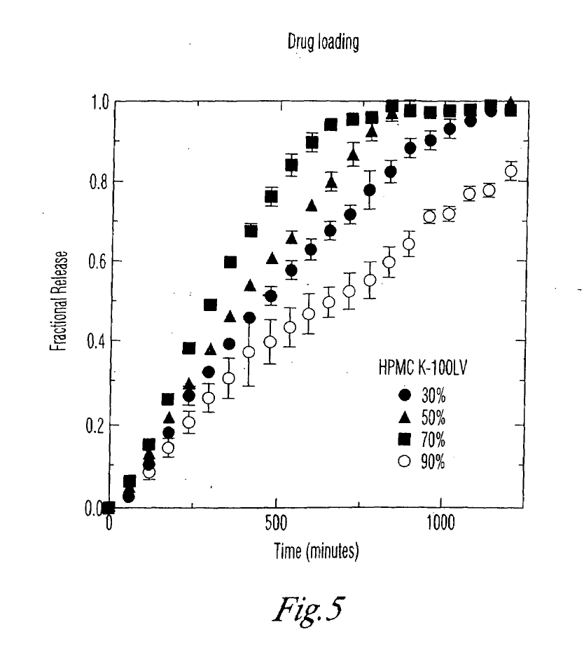

Next, tablets of diltiazem-deoxycholate were prepared with varying percentages of drug complex and HPMC K-100LV. The percent of drug complex in the tablets is shown in FIG. 5. All formulations released diltiazem with zero-order kinetics (FIG. 5). The fastest release was 70% diltiazem-deoxycholate and 30% HPMC K-100LV. The slowest was with 90% drug complex and 10% HPMC. The inventor believes the explanation of these data is that as the percent bile complex increases, the release rate increases up to a point. But at the highest percentages of bile complex, the release rate slows because the controlling mechanism shifts from polymer erosion to drug-bile complex dissolution.

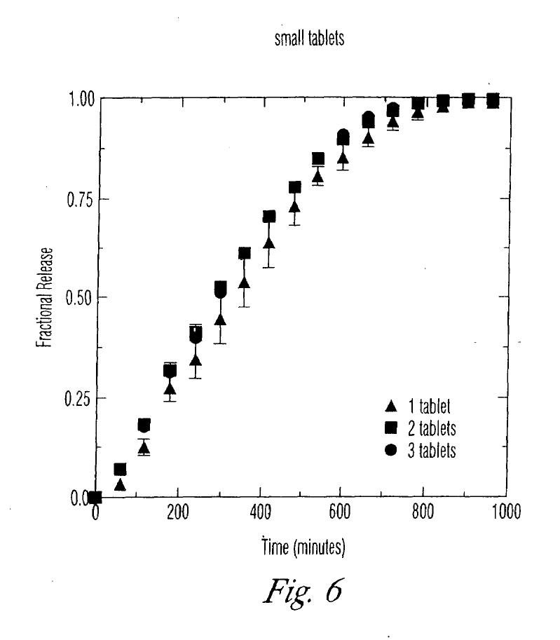

Next, the dependence of release kinetics on the number of tablets was tested. One, two, or three small tablets (150 mg) containing 50% diltiazem-deoxycholate and 50% HPMC 4M were tested. The fractional release kinetics were virtually identical regardless of the number of tablets (FIG. 6).

### Example 2

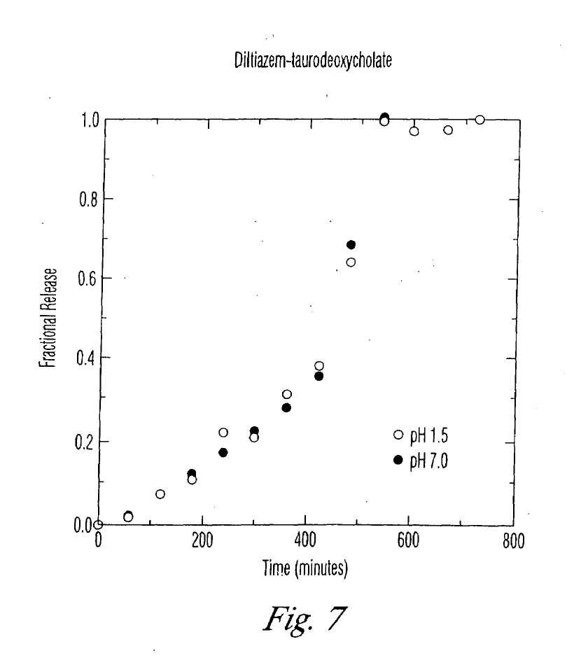

Diltiazem-taurodeoxycholate was prepared by precipitation from aqueous solution as described for diltiazem-deoxycholate in Example 1. The drug-bile complex was pressed into tablets without any binders or excipients as in Example 1. The release rate from a tablet in pH 1.5 and pH 7.0 aqueous solution is shown in FIG. 7. At both pHs the release proceeded slowly with zero-order kinetics until about 0.4 fractional release. From that point, the remaining drug was quickly released. This occurred because the tablets were rigid until fractional release of about 0.4, and after that time, the tablets broke up. Taurodeoxylcholate is more hydrophilic than deoxycholate and absorbs more water. This causes the tablets containing the taurodeoxycholate complexes to have less structural stability than tablets containing deoxycholate complexes. This problem can be overcome by adding a binder or polymer matrix to the tablets to improve their structural stability.

All patents, patent documents, and other references cited herein are incorporated by reference.

What is claimed is:

1-23. (canceled)

24. A method of preparing a sustained-release medicament comprising:
contacting an ionic small molecule pharmaceutically active agent, having a molecular weight of less than 2,000 and a solubility in water of at least 2 mg/ml, with an oppositely charged ionic surfactant to form a sustained-release ionic complex between the active agent and the surfactant to sustain release of the active agent; and
formulating the sustained-release ionic complex into a sustained-release medicament;
wherein the medicament is an ingestible tablet, buccal tablet, troche, or capsule.

25. The method of claim 24 wherein the contacting in aqueous solution forms a solid precipitate sustained-release ionic complex, which is formulated into a sustained-release medicament without redissolution in a solvent with a polymer matrix and precipitation in the polymer matrix.

26. The method of claim 24 wherein the sustained-release medicament releases the ionic pharmaceutically active agent into solution with zero-order kinetics in an aqueous solution containing salt.

27. The method of claim 24 wherein the pharmaceutically active agent is non-peptidyl.

28. The method of claim 24 wherein the surfactant is a naturally occurring molecule in humans.

29. The method of claim 24 wherein the surfactant is a bile anionic surfactant.

30. The method of claim 24 wherein the step of formulating the sustained-release complex into the sustained-release medicament comprises mixing or coating the sustained-release complex with a sustained-release polymer filler or coating to form a polymer-containing sustained-release medicament.

31. The method of claim 30 wherein the sustained-release polymer is hydroxypropylmethylcellulose, polyethylene oxide, hydroxypropylcellulose, hydroxyethylcellulose, methylcellulose, or a polysaccharide.

32. The method of claim 24 wherein the pharmaceutically active agent is cationic and is diltiazem, propranolol, verapamil, lebatalol, setraline, venlafaxine, clopidogrel, amlodipine, fexofenadine, or bupropion.

33. The method of claim 24 wherein the pharmaceutically active agent is anionic and is atorvastatin, esomerprazole, montelukast, pravastatin, alendronate, levothyroxine, or risedronate.

34. The method of claim 24 wherein the ionic pharmaceutically active agent has a solubility in water of at least 40 mg/ml.

35-39. (canceled)

40. The method of claim 24 wherein the contacting is in aqueous solution and the ionic sustained release complex forms as a precipitate.

41. The method of claim 24 wherein the medicament is a tablet.

42. The method of claim 40 wherein the medicament is a tablet.
